(19) United States
(12) Patent Application Publication
(10) Pub. No.: US 2014/0088457 A1
(43) Pub. Date: Mar. 27, 2014

(54) BLEEDING CONTAINMENT DEVICE

(71) Applicant: Covidien LP, Mansfield, MA (US)

(72) Inventor: Jay G. Johnson, Maple Plain, MN (US)

(73) Assignee: Covidien LP, Mansfield, MA (US)

(21) Appl. No.: 14/037,595

(22) Filed: Sep. 26, 2013

Related U.S. Application Data
(60) Provisional application No. 61/705,713, filed on Sep. 26, 2012.

(51) Int. Cl. A61B 17/12; A61B 10/04

(52) U.S. Cl. CPC A61B 17/12136; A61B 10/04
USPC 600/567; 606/192

(57) ABSTRACT

A system configured for use in an anatomical luminal network of a patient includes an extended working channel and a navigation system for guiding one or more tools, the extended working channel, or a locatable guide through a luminal network following a predetermined pathway to target tissue. The extended working channel is configured to provide passage for a tool. The extended working channel includes a bleeding containment device including a balloon portion and an inflation duct.

### CROSS-REFERENCE TO RELATED APPLICATIONS

The present application claims priority to, and the benefit of, U.S. Provisional Application Ser. No. 61/705,713, filed on Sep. 26, 2012, the disclosure of which is herein incorporated by reference in its entirety.

### BACKGROUND

1. Technical Field

The present invention relates to methods and devices for use in performing medical procedures including delivery devices and procedures for the lungs. More particularly, the present disclosure relates to bleeding containment devices and methods of containing bleeding within the lungs during a medical procedure.

2. Discussion of Related Art

Endoscopes have been in common use in medical practice for many years. Flexible endoscopes enable surgeons to carry out a variety of diagnostic and therapeutic procedures. The bronchoscope, which is a type of endoscope, is a flexible tube often having a fiberoptic bundle end or video camera located at the distal tip. Actuated from a handle at its proximal end, the tip is deflectable in one or more directions to allow the device to be steered inside the bronchial tree during insertion to permit proper positioning. A typical bronchoscope is a two or three-lumen structure including a working channel that allows tools to be inserted into the channel at the handle and through the length of the bronchoscope to the tip.

Clinicians obtain biopsy specimens for various purposes such as diagnosing, staging and grading disease states. A biopsy procedure may be performed using needles, bronchoscopes, and/or surgery to obtain tissue or fluid samples. The appropriate method of biopsy for a patient depends upon a variety of factors, including the size, location, appearance and characteristics of the abnormality, and the patient's medical history. The clinician performing the biopsy may use specialized imaging equipment to guide the biopsy tool to the desired site. Some biopsies may be performed under image guidance using ultrasound, computed tomography (CT) scan, or magnetic resonance imaging (MRI). For example, CT imaging may be used to guide lung biopsies.

Potential complications in medical procedures include bleeding during surgery. One concern of doctors during pulmonary surgery or drug delivery is bleeding within the lungs. Bleeding within the lungs can be monitored with the camera and light at the end of the bronchoscope, but the bronchoscope does not allow viewing of tissue within passageways too narrow for the bronchoscope to enter.

Bleeding within the lungs is a risk with procedures like a needle biopsy procedure, in which a flexible catheter with a biopsy needle at its distal tip is inserted into the working channel of a bronchoscope and punctured through the wall of the pulmonary passageway to the center of the lesion. This procedure may involve challenges because vital organs such as large blood vessels can be damaged if accidentally pierced by the biopsy needle. Placement of the needle may be enhanced by the use of image guidance. For example, guiding the needle according to three-dimensional (3D) imaging data such as computer tomography (CT) data may help to avoid vessel damage. Unfortunately, there is no guarantee that the use of imaging modalities for guidance will prevent bleeding within the lungs.

PCT application published as WO 03/086498 entitled “Endoscopic Structures and Techniques for Navigating to a Target in Branched Structure” to Gilboa, which is herein incorporated by reference in its entirety, describes methods and apparatuses in which a thin locatable guide, enveloped by a sheath or extended working channel, is used to navigate a bronchoscopic tool to a target location within the lung, aimed in particular to deliver treatments to the lung periphery beyond the bronchoscope's own reach. The coordinates of the target are predetermined based upon three-dimensional CT data. A location sensor is incorporated at the locatable guide's tip. The locatable guide is inserted into the lung via the working channel of a bronchoscope. First, the bronchoscope's tip is directed to the furthest reachable location in the direction of the target. The passageways of the bronchial tree become progressively narrower as they branch with increasing depth into the bronchial tree. Narrower branched structures of the lungs cannot be reached by the bronchoscope. Once the bronchoscope is wedged in the lungs, the extended working channel and locatable guide is advanced beyond the tip of the bronchoscope towards the designated target, based on the combination of the CT data and the sensed position of the locatable guide. After bringing the locatable guide to the target, the locatable guide is withdrawn from the extended working channel and a bronchoscopic tool is inserted into the empty extended working channel up to the target. Navigation of the extended working channel may rely on a steering mechanism of the locatable guide due to insufficient room for a steering mechanism as part of the extended working channel. In order to prevent the distal end of the extended working channel from sliding away from the target, the extended working channel is locked to the bronchoscope's body and the bronchoscope itself is held steady by the clinician to prevent it from slipping.

A variety of biopsy techniques may be applied. After the biopsy specimen is obtained, the specimen is examined to determine if abnormal or cancerous cells are present.

### SUMMARY

There is a need to detect and contain bleeding within the lungs during surgery. A continuing need exists for devices to detect and/or monitor bleeding within the lungs during surgery. There is a need for devices to contain any bleeding within the lungs during surgery. A need exists for a device to detect and contain any bleeding within smaller regions of the lungs, while not compromising the available space to move within the lungs. There is a continuing need for biopsy devices.

According to an aspect of the present disclosure, a device is provided for use with a bronchoscope. The device includes an extended working channel configured to provide passage for a tool. The extended working channel and tool are sized to traverse a working channel of the bronchoscope. A bleeding containment device is associated with a distal end portion of the extended working channel. The bleeding containment device includes a balloon portion and an inflation duct associated with the extended working channel.

According to another aspect of the present disclosure, a system is provided configured for use in an anatomical luminal network of a patient. The system includes an extended working channel and a navigation system for guiding one or more tools, the extended working channel, or a locatable guide through a luminal network following a predetermined pathway to target tissue. The extended working channel is configured to provide passage for a tool. The extended working channel includes a bleeding containment device including a balloon portion.

According to another aspect of the present disclosure, a biopsy tool for use with an extended working channel is provided. The biopsy tool includes an elongated shaft including a distal end and a tissue-sampling device disposed at the distal end of the elongated shaft. The biopsy tool also includes a bleeding containment device associated with a distal end portion of the elongated shaft. The bleeding containment device includes a balloon portion and an inflation duct associated with the elongated shaft.

According to another aspect of the present disclosure, a method of containing bleeding within the lungs is provided. The method includes the initial step of providing an extended working channel configured to provide passage for a tool, wherein the extended working channel includes a bleeding containment device including a balloon portion. The method also includes the steps of navigating the extended working channel including the bleeding containment device to a target site within a pulmonary airway of a patient, inflating the balloon portion, which anchors a distal end of the extended working channel within the pulmonary airway at the target site, and advancing the tool within the extended working channel to the target site.

### BRIEF DESCRIPTION OF THE DRAWINGS

Objects and features of the presently-disclosed bleeding containment devices, extended working channels including a bleeding containment device, and methods of containing bleeding within the lungs using the same will become apparent to those of ordinary skill in the art when descriptions of various embodiments thereof are read with reference to the accompanying drawings, of which:

FIG. 1 is a perspective view of a navigation system for use in an anatomical luminal network of a patient, such as the pulmonary airways, configured for use with a bronchoscope in accordance with an embodiment of the present disclosure;

FIG. 2A is a schematic view of a portion of the bronchoscope shown in FIG. 1 and an extended working channel including a bleeding containment device with a portion of the extended working channel positioned within the bronchoscope prior to being positioned within the trachea of the patient in accordance with an embodiment of the present disclosure;

FIG. 2B is a schematic view of the bronchoscope shown in FIG. 2A positioned within the trachea of the patient and the extended working channel including the bleeding containment device positioned within a lung of the patient with the balloon portion of the bleeding containment device in a deflated configuration in accordance with an embodiment of the present disclosure;

FIG. 3 is a schematic view of the bronchoscope and the working channel shown in FIG. 2B with the extended working channel positioned within the lung of the patient, showing the balloon portion of the bleeding containment device in an inflated configuration, in accordance with an embodiment of the present disclosure;

FIG. 4 is an enlarged view of the area of detail indicated in FIG. 3 illustrating a portion of the extended working channel including the bleeding containment device with the balloon portion thereof in an inflated configuration within the lung's periphery in accordance with an embodiment of the present disclosure;

FIG. 5 is a schematic view of the bronchoscope shown in FIG. 1 positioned within the trachea of the patient with the extended working channel including the bleeding containment device positioned within a lung of the patient, shown with a tool, e.g., a biopsy tool, prior to being advanced within the extended working channel, in accordance with an embodiment of the present disclosure;

FIG. 6 is an enlarged, cross-sectional view of the extended working channel including a bleeding containment device and the biopsy tool shown in FIG. 5 positioned within the bronchoscope, shown with a portion of a tool positioned distally from the distal end of the extended working channel, in accordance with an embodiment of the present disclosure;

FIG. 7 is an enlarged view of the area of detail indicated in FIG. 5 illustrating a portion of the extended working channel including with the balloon portion of the bleeding containment device in a deflated configuration within a pulmonary airway in accordance with an embodiment of the present disclosure;

FIG. 8 is an enlarged view of the area of detail indicated in FIG. 4 illustrating a portion of the extended working channel with the balloon portion of bleeding containment device in a deflated configuration within a pulmonary airway in accordance with an embodiment of the present disclosure;

FIG. 9 is an enlarged view of an activation device for use with an extended working channel including a bleeding containment device in accordance with an embodiment of the present disclosure;

FIG. 10 is a schematic view of the bronchoscope shown in FIG. 1 positioned within the trachea of the patient with an extended working channel positioned within a lung of the patient, shown with a bleeding containment device prior to being advanced within the extended working channel, in accordance with an embodiment of the present disclosure;

FIG. 11 is an enlarged, cross-sectional view of the extended working channel and the bleeding containment device shown in FIG. 10 with a portion of extended working channel positioned within the bronchoscope, showing a balloon portion of the bleeding containment device positioned distally from the distal end of the extended working channel, in accordance with an embodiment of the present disclosure;

FIG. 12 is a schematic view of the bronchoscope shown in FIG. 1 positioned within the trachea of the patient with an extended working channel positioned within a lung of the patient, shown with a biopsy tool including a bleeding containment device prior to being advanced within the extended working channel, in accordance with an embodiment of the present disclosure;

FIG. 13 is an enlarged, cross-sectional view of the extended working channel and the bleeding containment device shown in FIG. 12 with a portion of extended working channel positioned within the bronchoscope, showing a balloon portion of the bleeding containment device positioned distally from the distal end of the extended working channel, in accordance with an embodiment of the present disclosure;

FIG. 14 is an enlarged cross-section view taken along the lines 14-14 of FIG. 6;

FIG. 15 is an enlarged cross-section view taken along the lines 15-15 of FIG. 6;

FIG. 16 is an enlarged cross-section view taken along the lines 16-16 of FIG. 6;

FIG. 17 is an enlarged cross-section view taken along the lines 17-17 of FIG. 6; and

FIG. 18 is a flowchart illustrating a method of containing bleeding within the lungs in accordance with an embodiment of the present disclosure.

### DETAILED DESCRIPTION

Hereinafter, embodiments of the presently-disclosed bleeding containment device, extended working channel including a bleeding containment device, and method of containing bleeding within the lungs using the same are described with reference to the accompanying drawings. Like reference numerals may refer to similar or identical elements throughout the description of the figures. As shown in the drawings and as used in this description, and as is traditional when referring to relative positioning on an object, the term “proximal” refers to that portion of the device, or component thereof, closer to the user and the term “distal” refers to that portion of the device, or component thereof, farther from the user.

This description may use the phrases “in an embodiment,” “in embodiments,” “in some embodiments,” or “in other embodiments,” which may each refer to one or more of the same or different embodiments in accordance with the present disclosure.

As it is used herein, the term “patient” generally refers to a human or other animal. For the purposes of this description, the term “fluid” generally refers to a gas, a liquid, or a mixture of gas and liquid. As it is used herein, the term “beneficial agent” generally refers to any physiologically active substance(s), pharmacologically active substance(s), and/or psychotropic substance(s).

As it is used in this description, “transmission line” generally refers to any transmission medium that can be used for the propagation of signals from one point to another. A transmission line may be, for example, a wire, a two-wire line, a coaxial wire, and/or a waveguide.

The term “user interface” as used herein refers to an interface between a human user or operator and one or more devices that enables communication between the user and the device(s). Examples of user interfaces that may be employed in various implementations of the present invention include without limitation display screens, various types of graphical user interfaces (GUIs), touch screens, microphones and other types of sensors that may receive some form of human-generated stimulus and generate a signal in response thereto. For the purposes of this description, the term “code” should be interpreted as being applicable to software, firmware, or a combination of software and firmware.

Various embodiments of the present disclosure provide devices and methods for contain bleeding within the lungs while allowing lung surgery to take place effectively. Various embodiments of the present disclosure provide a navigation system for use in an anatomical luminal network, e.g., the pulmonary airways, including an extended working channel to achieve access to an identified target. In some embodiments, the presently-disclosed extended working channel includes a bleeding containment device including a balloon portion disposed proximate to the distal end of the extended working channel.

Various embodiments of the present disclosure provide a bleeding containment device adapted for use with an extended working channel and configured to be slideably moveable within the extended working channel. Various embodiments of the present disclosure provide a biopsy tool including a bleeding containment device configured for use with an extended working channel.

Various embodiments of the present disclosure provide biopsy tools including a tissue-sampling device, e.g., end-effectors, micro-forceps, micro-nozzles, and absorbent materials. In some embodiments, the biopsy tools include drug delivery (and/or contrast agent) components and biopsy tissue-sampling components.

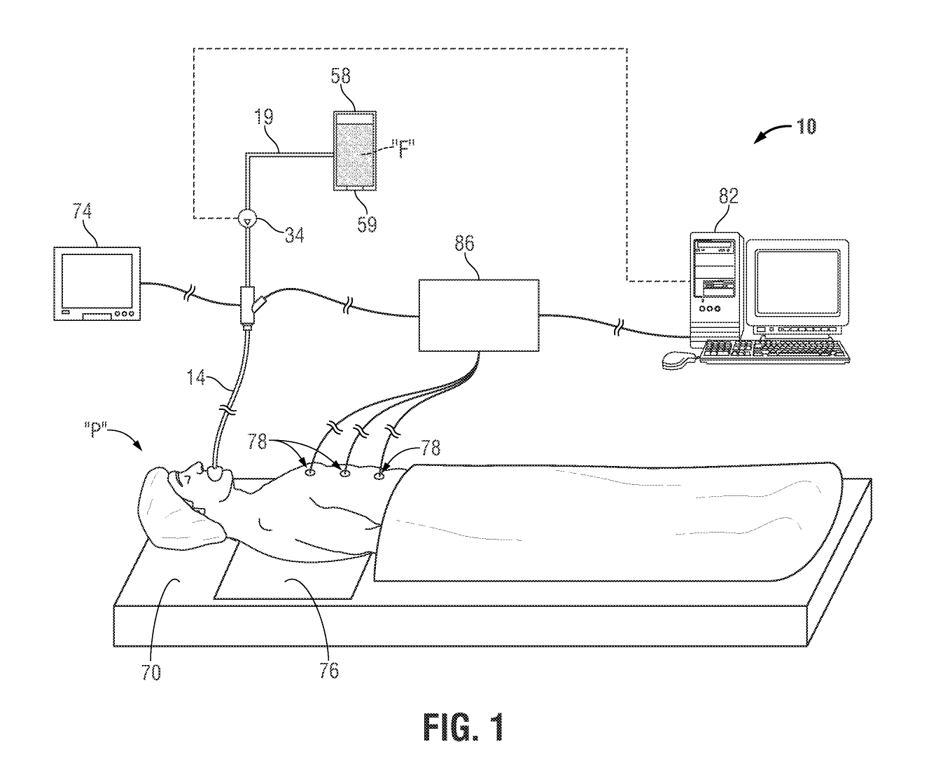

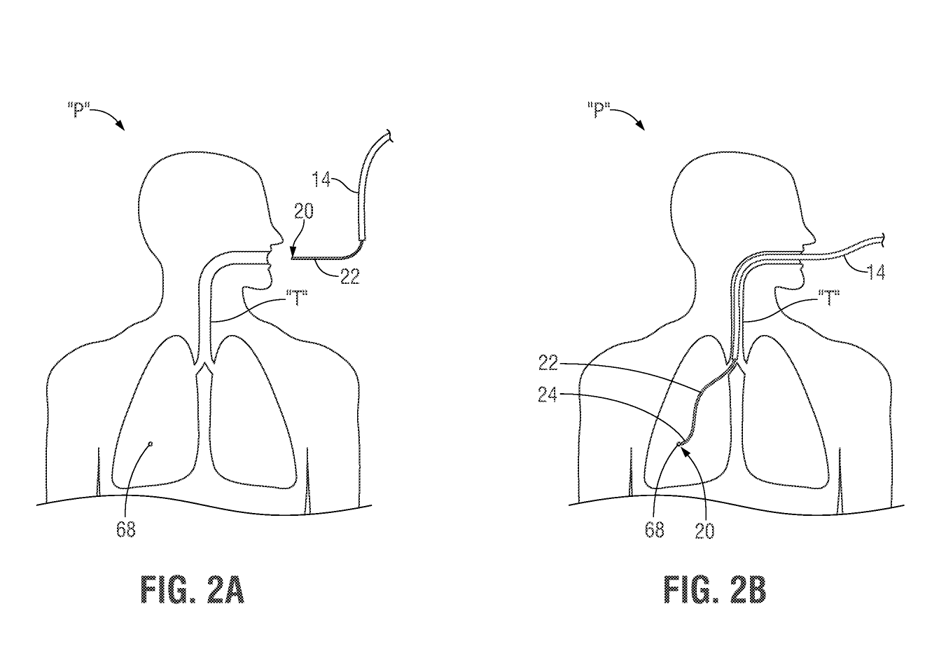

FIG. 1 shows a patient “P” lying on an operating table 70 in a supine position and connected to a navigation system 10 adapted for use in an anatomical luminal network, such as the pulmonary airways. System 10 is adapted to enable navigation along a determined pathway within the luminal network to achieve access to an identified target. In some embodiments, the system 10 is adapted to analyze a three-dimensional model of the luminal network and automatically determine a pathway from an entry point to a designated target (e.g., target 68 indicated by the circle shown in FIGS. 2A and 2B). System 10 may be further adapted to automatically assign waypoints along the determined pathway in order to assist a physician in navigating a catheter device or “extended working channel” to the designated target.

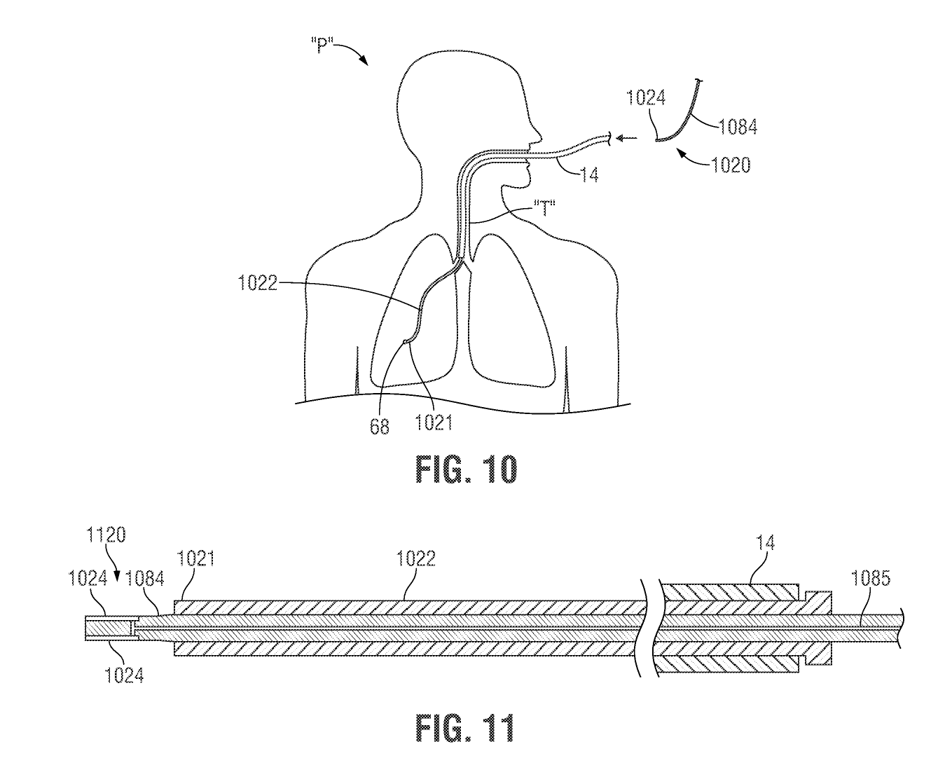

As shown in FIG. 1, a bronchoscope 14 is connected to monitoring equipment 74. A display and/or user interface may be provided to assist the user in navigating to the target. Bronchoscope 14 is inserted into the patient's lungs, and may include a source of illumination and a video imaging system. A catheter or extended working channel (e.g., extended working channel 22 shown in FIGS. 2A and 2B, or extended working channel 1022 shown in FIGS. 10 and 11) is positioned, in part, within the bronchoscope 14.

System 10 monitors the position of the patient “P,” thereby defining a set of reference coordinates. System 10 utilizes an electromagnetic navigation system, such as that described in the PCT application published as WO 03/086498 discussed above. In some embodiments, the system 10 may additionally, or alternatively, utilize a six degrees-of-freedom electromagnetic position measuring system according to the teachings of U.S. Pat. No. 6,188,355 entitled “Wireless Six-Degree-of-Freedom Locator” to Gilboa, and published PCT application nos. WO 00/10456 and WO 01/67035, which are herein incorporated by reference in their entireties.

A transmitter arrangement 76 is implemented as a board or mat positioned beneath the patient “P.” A plurality of sensors 78 are interconnected with a tracking module 86 which derives the location of each sensor 78 in six degrees-of-freedom. One or more of the sensors 78 (e.g., 3 sensors 78) are attached to the chest of patient “P” and their six degrees-of-freedom coordinates sent to a computer 82 where the coordinates are used to calculate the patient coordinate frame of reference. The computer 82 may include any type of computing device, computational circuit, or any type of processor or processing circuit capable of executing a series of instructions that are stored in a memory (not shown) associated with the computer 82. Computer 82 may be adapted to run an operating system platform and application programs. In some embodiments, data and/or code may be stored in a library (not shown) communicatively coupled to computer 82. As it is used in this description, “library” generally refers to any repository, databank, database, cache, storage unit and the like.

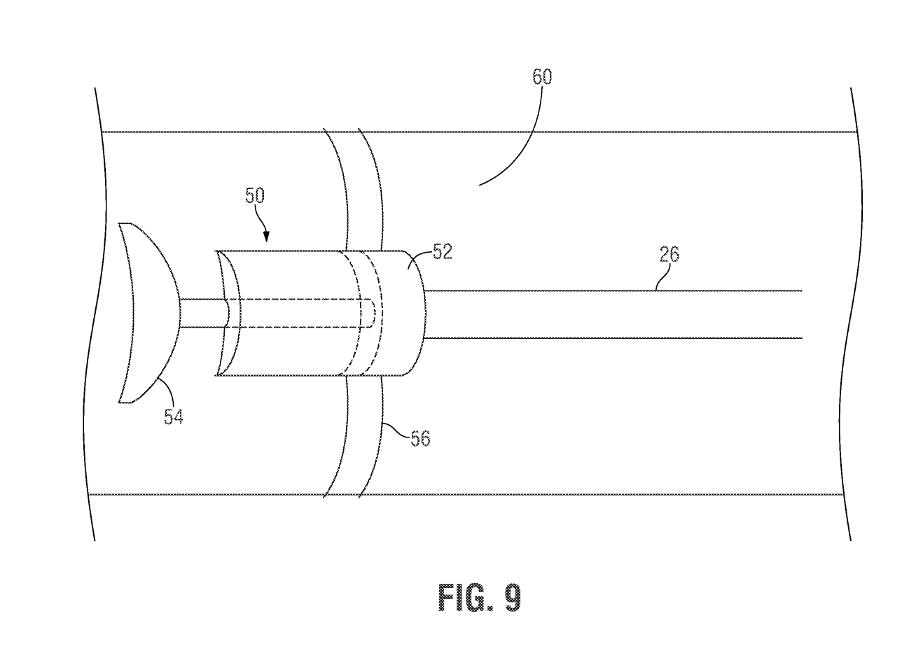

In some embodiments, as shown in FIG. 1, system 10 includes a fluid reservoir 58 and a fluid path 19 leading from the fluid reservoir 58 to the bronchoscope 14 and/or the extended working channel associated therewith. Additionally, or alternatively, an activation device 50 (shown in FIG. 9) may be provided for use with the extended working channel.

Fluid reservoir 58 includes any suitable housing containing a reservoir of fluid “F.” Various fluids may be used, e.g., liquids including, but not limited to, water and saline. In other variations, gases (such as nitrous oxide, nitrogen, carbon dioxide, etc.) may also be utilized as the fluid “F.” In yet another variation, a combination of liquids and/or gases, including, for example, those mentioned above, may be utilized as the fluid “F.” In some embodiments, the fluid reservoir 58 may include a temperature control device 59 adapted to maintain fluid “F” at a predetermined temperature.

As shown in FIG. 1, a fluid movement device 34 may be provided in the fluid path 19 to move the fluid “F” through the fluid path 19. Fluid movement device 34 may include valves, pumps, power units, actuators, fittings, manifolds, etc. In some embodiments, the fluid movement device 34 may be communicatively coupled via a transmission line (as indicated by the dashed line in FIG. 1) to the computer 82, which may be configured to control one or more operating parameters of the fluid movement device 34. The position of the fluid movement device 34 may be varied from the configuration depicted in FIG. 1.

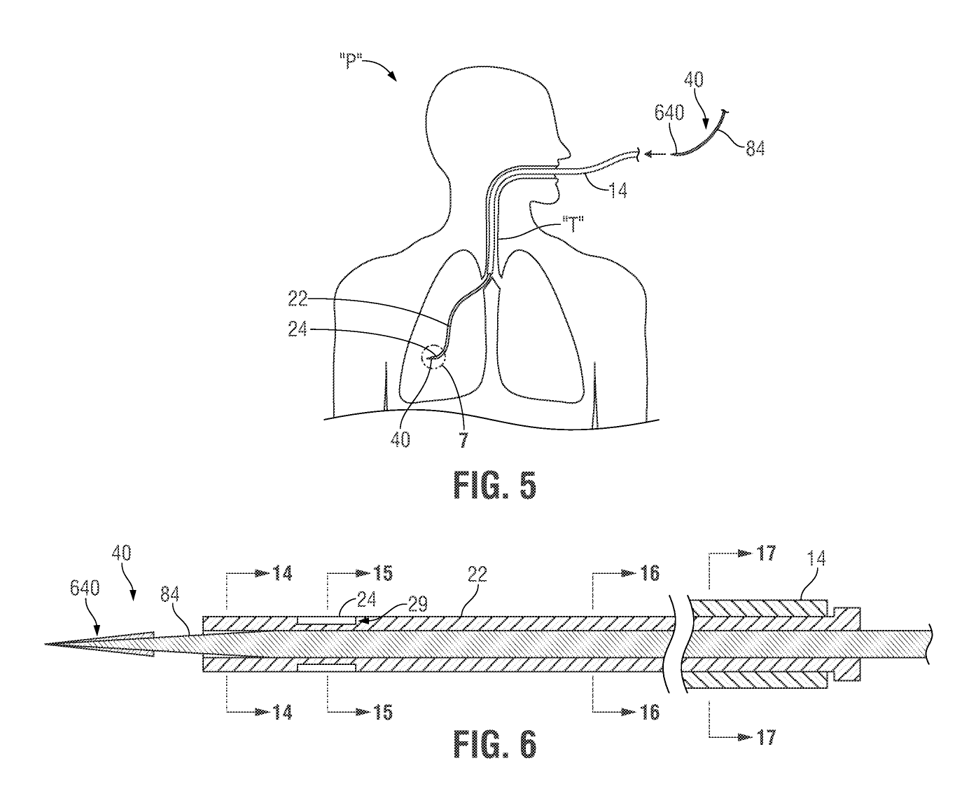

In an embodiment, such as, for example, when the target 68 is relatively close to the distal end of the bronchoscope 14, the extended working channel 22 may be removed, or not used at all, and the bronchoscope 14 kept in place to visually guide tools to the target 68. Alternatively, the extended working channel 22 and accompanying access tools may be placed without use of the bronchoscope 14, or the bronchoscope 14 can be removed after placement of the extended working channel 22 in combination with access tools at the target 68 and kept in place and a tool (e.g., tool 40 shown in FIG. 5) can be extended through the extended working channel 22 to treat the target 68.

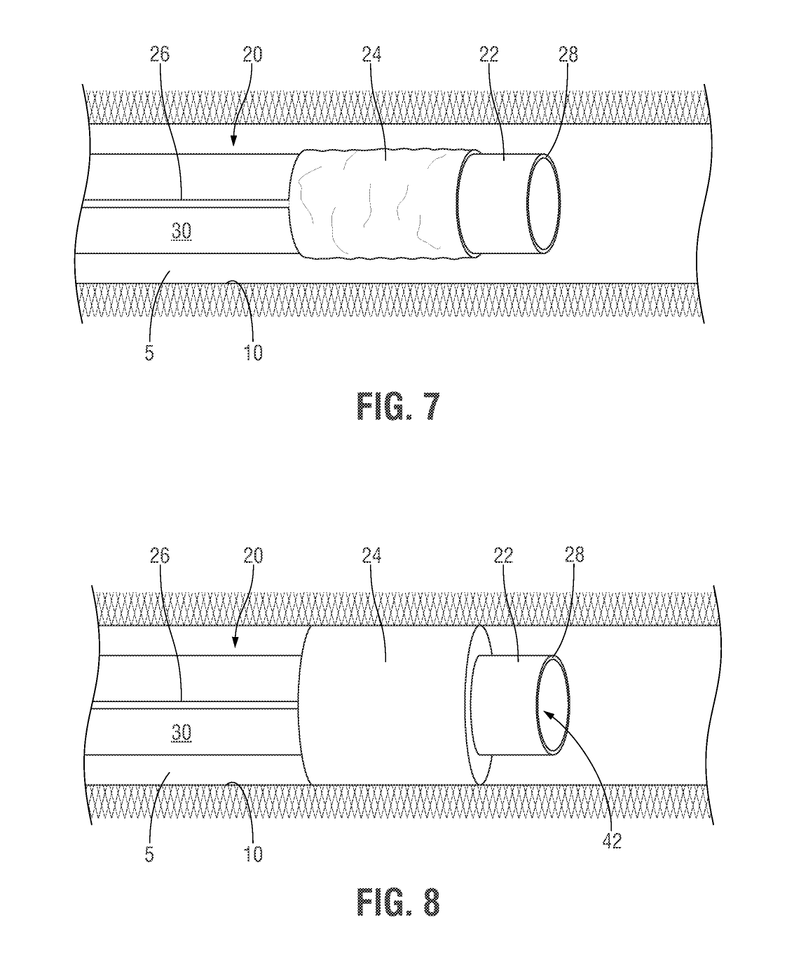

FIG. 2A shows a portion of the bronchoscope 14 shown in FIG. 1 and a flexible, thin catheter or an extended working channel 22 including a bleeding containment device 20, wherein a portion of the extended working channel 22 is positioned within the bronchoscope 14 prior to being positioned within the trachea “T” of the patient “P.” The bleeding containment device 20 generally includes a balloon portion 24 coupled to the extended working channel 22 and an inflation duct (e.g., inflation duct 26 shown in FIGS. 7 and 8) configured to allow fluid “F” (FIG. 1) to flow to and from the balloon portion 24 for inflation and deflation purposes.

The bleeding containment device 20 may be configured in a delivery configuration, wherein the balloon portion 24 is deflated. In some embodiments, the balloon portion 24 is configured to lie relatively flat against the extended working channel 22. In some embodiments, as shown in FIG. 7, balloon portion 24 minimally increases the diameter of the extended working channel 22. In some embodiments, as shown in FIGS. 6 and 15, the outer surface of the extended working channel 22 includes a recess 29 defined therein configured to receive the balloon portion 24 such that the outer circumference of the balloon portion 24 is substantially the same as the outer circumference of the extended working channel 22.

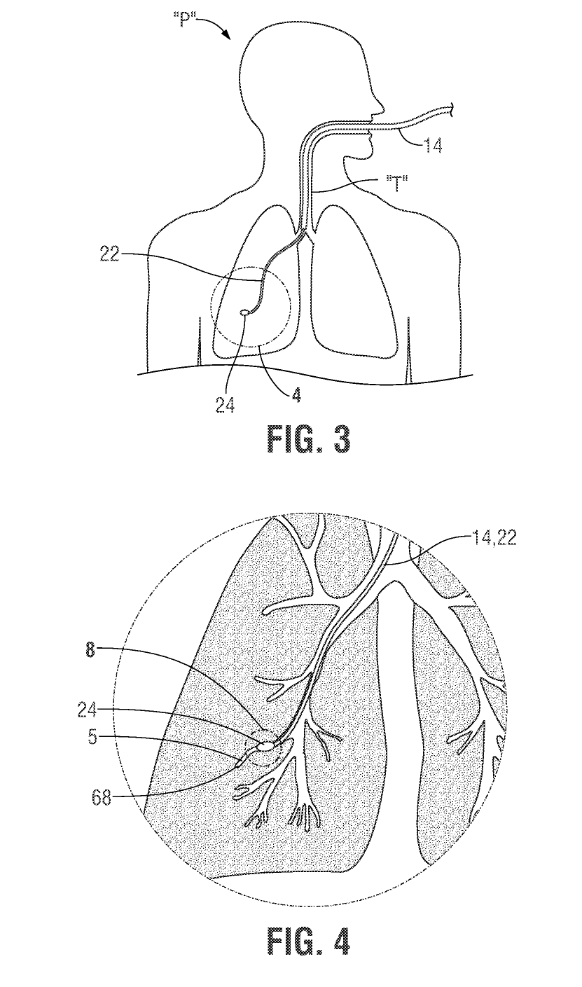

In operation, the bleeding containment device 20 can be used in a variety of ways. For example, in one embodiment, the extended working channel 22 including the bleeding containment device 20 is navigated to a target site within the pulmonary airways (e.g., pulmonary airway 5 shown in FIG. 4). Once the user is satisfied with the position of the distal end of the extended working channel 22, the user may inflate the balloon portion 24, which will then act to anchor the distal end of the extended working channel 22 within the airways in the desired location. A tool 40 (FIG. 6) may then be advanced through the lumen 28 of the extended working channel 22 to perform a desired procedure on surrounding tissue. Upon completion of the procedure, the balloon portion 24 may be deflated and the bleeding containment device 20 may be retracted from the lungs or navigated to a next target location and re-inflated.

Alternatively, referring to FIGS. 7 and 8, the balloon 24 of the bleeding containment device 20 may be used only when needed. For example, the bleeding containment device 20 is used as an extended working channel to provide a conduit for various target locations, leaving the balloon portion 24 in a deflated mode. In the event bleeding occurs, the balloon portion 24 is inflated via the inflation duct 26, thereby applying direct pressure to the area that is bleeding. The bleeding containment device 20 may be kept in place for a time sufficient for coagulants in the blood stream to stop the bleeding. Alternatively, the containment device 20 may create a seal against the walls 10 of the airways 5 surrounding the balloon portion 24. Once sealed, blood is blocked from traveling proximally and can be aspirated through the interior lumen 28 of the extended working channel 22 (e.g., as indicated by arrow 42 in FIG. 8).

FIG. 2B shows the bronchoscope positioned within the trachea “T” of the patient “P” with the extended working channel 22 including the bleeding containment device 20 positioned within a lung of the patient “P.” In FIG. 2B, the balloon portion 24 is illustratively depicted in a deflated configuration.

FIG. 3 shows the bronchoscope 14 and the extended working channel 22 positioned within the trachea “T” of the patient “P” with a portion of the extended working channel positioned within a lung of the patient “P”. In FIG. 4, a portion of the extended working channel 22 is shown positioned within a pulmonary airway 5 in the lung's periphery, wherein the balloon portion 24 of the bleeding containment device 20 is disposed in an inflated configuration.

FIG. 5 shows the bronchoscope of FIG. 1 positioned within the trachea “T” of the patient “P” with the extended working channel 22 including the bleeding containment device 20 positioned within a lung of the patient “P.” In FIG. 5, a tool 40 (e.g., a biopsy tool) is shown prior to being advanced within the extended working channel 22.

As shown in FIG. 6, the tool 40 includes an elongated shaft 84 and a tissue-sampling device 640 disposed at the distal end of the shaft 84. In some embodiments, the tissue-sampling device includes a camera device (not shown). The camera device may include a micro-lens and a CCD, CMOS or other imaging device.

FIG. 7 shows a portion of the extended working channel 22 with the balloon portion 24 of the bleeding containment device 20 in a deflated configuration within a pulmonary airway 5. The inflation duct 26 is configured to extend over the length the extended working channel 22 from its proximal end to the balloon portion 24. In some embodiments, as shown in FIGS. 7 and 8, the inflation duct 26 is located on an outside surface 30 of the extended working channel 22, e.g., in order to maximize the potential use of the interior lumen 28 of the extended working channel 22. Locating the inflation duct 26 on the outside surface 30 may also prevent inadvertent crimping of the duct 26 when a tool 40 is advanced through the extended working channel 22. In some embodiments, the outside surface 30 of the extended working channel 22 includes a longitudinally extending recess or groove configured to receive at least a portion of the inflation duct 26 therein. In other embodiments, the inflation duct 26 may be located on an inside surface of the extended working channel 22, e.g., to lessen the possibility that the bleeding containment device 20 may be a tight fit within a working channel of the bronchoscope 14.

FIG. 9 shows an activation device 50 for use with the presently-disclosed extended working channel 22 including a bleeding containment device 20 shown in FIGS. 7 and 8. The activation device 50 generally includes a reservoir 52 and a plunger 54 slidingly arranged within the reservoir 52 for changing a volume thereof. The reservoir 54 is disposed in fluid communication with the inflation duct 26 and contains, along with the duct 26, a sufficient volume of fluid to inflate the balloon portion 24. The plunger 54 is depressed in order to inflate the balloon portion 24 and retracted to deflate the balloon 24. The plunger 54 may be spring-loaded and include a release mechanism in the event that the balloon portion 24 is to be inflated only when bleeding is detected. This embodiment provides a fast response to detected bleeding. In some embodiments, as shown in FIG. 9, activation device 50 has a crescent shape such that it may be attached to an outside of a bronchoscope handle 60 with an attachment mechanism 56, e.g., a strap, hook and loop fastener, temporary adhesive, etc. The shape and size of the activation device 50 may be varied from the configuration shown in FIG. 9.

Because the device 20 is to be used in the airways, it is feasible to use a gas as the fluid in the reservoir 52. As such, the gas could be pressurized slightly, e.g., pressurized enough to inflate the balloon 24 to a desired pressure. In the event pressurized gas is used as the fluid, the activation mechanism may include a bladder isolating the pressurized reservoir from the inflation duct 26 until inflation of the balloon 24 is desired. Inflation may be effected by providing a puncturing mechanism, e.g., arranged like the plunger 54 shown in FIG. 9, which, when depressed, pokes a hole in the membrane. When deflation is desired, a relief valve may be operated to release the pressure in the inflation duct 26. Pressurized gas may provide a fast activation time but may be temperature dependent and/or may be limited to a single use.

FIG. 10 shows the bronchoscope shown in FIG. 1 positioned within the trachea “T” of the patient “P” with a catheter or extended working channel 1022 positioned within a lung of the patient “P.” In FIG. 10, a bleeding containment device 1020 is shown prior to being advanced within the extended working channel 1022. The bleeding containment device 1020 includes an elongated shaft 1084 and a balloon portion 1024 disposed at the distal end of the shaft 1084.

In some embodiments, as shown in FIG. 11, bleeding containment device 1020 includes an inflation duct 1085 disposed within the shaft 1084. The inflation duct 1085 is configured to provide fluid communication between the balloon portion 1024 and the proximal end of the shaft 1084. FIG. 11 shows the extended working channel 1022 and the bleeding containment device shown in FIG. 10 positioned within the bronchoscope 14 with the balloon portion 1024 of the bleeding containment device 1120 positioned distally from the distal end 1021 of the extended working channel 1022.

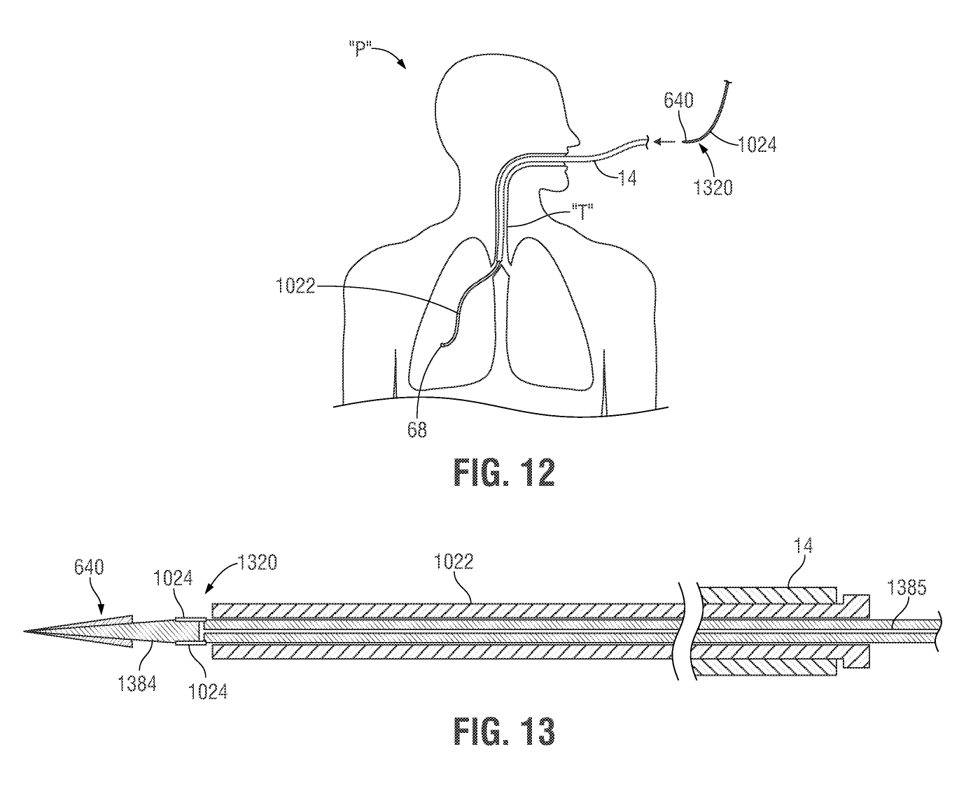

FIG. 12 shows the bronchoscope shown in FIG. 1 positioned within the trachea “T” of the patient “P” with an extended working channel 1022 positioned within a lung of the patient “P.” In FIG. 12, a tool including the tissue-sampling device 640 shown in FIG. 6 and a bleeding containment device 1320 is shown prior to being advanced within the extended working channel 1022, in accordance with an embodiment of the present disclosure. The bleeding containment device 1320 is similar to the bleeding containment device 1120 shown in FIG. 11, and further description thereof is omitted in the interests of brevity.

FIG. 13 shows the extended working channel and the bleeding containment device shown in FIG. 12, wherein a portion of the extended working channel 1022 is positioned within the bronchoscope 14, shown with the balloon portion 1024 of the bleeding containment device 1320 positioned distally from the distal end of the extended working channel 1022.

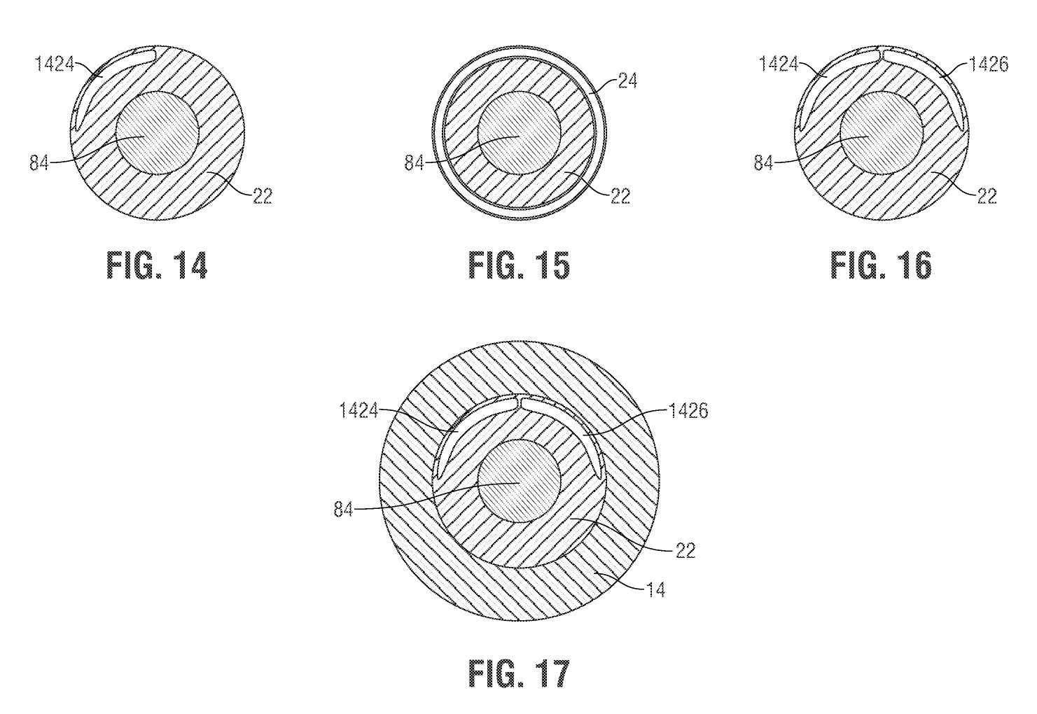

FIGS. 14 through 17 show enlarged, cross-sectional views of the extended working channel 22 including the bleeding containment device 20 of FIG. 6 showing the shaft 84 of the tool 40 disposed within the extended working channel 22.

FIG. 14 shows a cross-sectional view of the distal end of the extended working channel 22 including the bleeding containment device 20 of FIG. 6. In some embodiments, as shown in FIG. 14, the extended working channel 22 includes a lumen 1424 configured to allow delivery of a beneficial agent to the target site and/or to allow for suction at the target site. The lumen 1424 may be used for advancing a fiberoptic bundle or video camera to the target site. The shape and size of the lumen 1424 may be varied from the configuration shown in FIG. 14.

FIG. 15 shows a cross-sectional view of a portion of the extended working channel 22 including the balloon portion 24 of the bleeding containment device 20 of FIG. 6. In some embodiments, the wall of the balloon portion 24 may include a porous material, e.g., to allow delivery of a beneficial agent included in the fluid (e.g., fluid “F” shown in FIG. 1) used to inflate the balloon portion 24, e.g., in order to promote blood clotting.

FIG. 16 shows a cross-sectional view of a portion of the extended working channel 22 proximal to the balloon portion 24 of the bleeding containment device of FIG. 6. In some embodiments, as shown in FIG. 16, the extended working channel 22 includes an inflation duct 1426 configured to allow fluid to flow to and from the balloon portion 24 for inflation and deflation of the balloon portion 24.

FIG. 17 shows a cross-sectional view of a portion of the extended working channel 22 of FIG. 6 disposed within the bronchoscope 14.

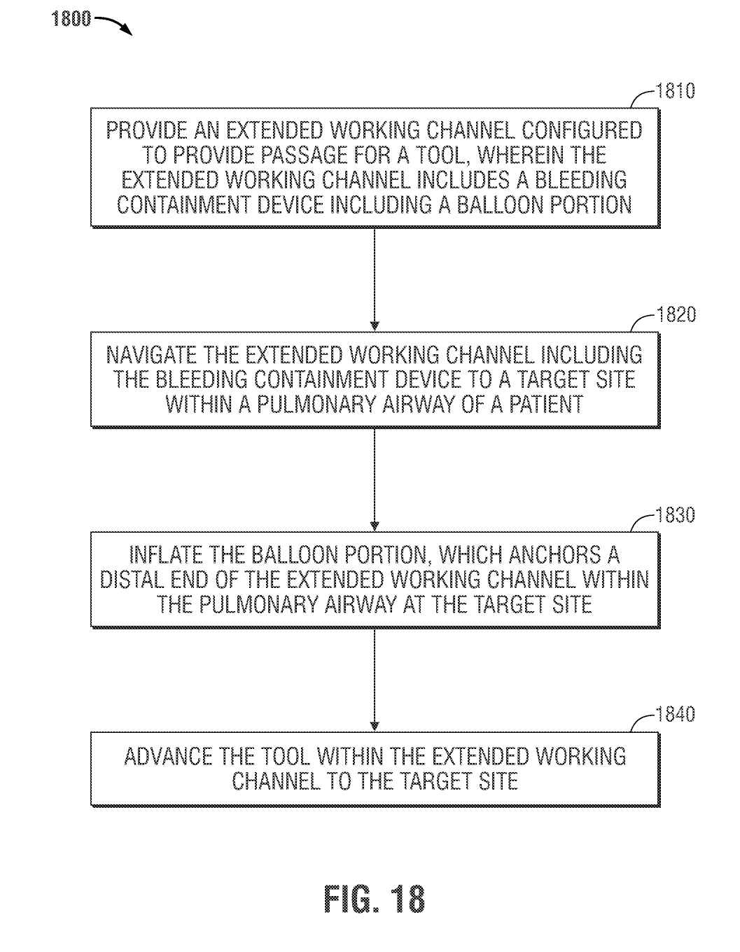

Hereinafter, a method of containing bleeding within the lungs, in accordance with the present disclosure, is described with reference to FIG. 18. It is to be understood that the steps of the method provided herein may be performed in combination and in a different order than presented herein without departing from the scope of the disclosure.

FIG. 18 is a flowchart illustrating a method of containing bleeding within the lungs according to an embodiment of the present disclosure. In step 1810, an extended working channel 22 configured to provide passage for a tool 40 is provided wherein the extended working channel 22 includes a bleeding containment device 20 including a balloon portion 24. In some embodiments the extended working channel 22 may include one or more lumens (e.g., lumen 1424 shown in FIG. 14 and/or lumen 1426 shown in FIG. 16), which may extend through most or all of the length of the extended working channel 22.

In alternative embodiments of a method of containing bleeding within the lungs in accordance with the present disclosure, wherein the extended working channel does not include the bleeding containment device 20, any of the presently-disclosed bleeding containment device embodiments (e.g., bleeding containment device 1120 shown in FIG. 11 or bleeding containment device 1320 shown in FIG. 13) may be advanced within the extended working channel.

In step 1820, the extended working channel 22 including the bleeding containment device 20 is navigated to a target site 68 within a pulmonary airway 5 of a patient “P.”

In step 1830, the balloon portion 24 is inflated, which anchors a distal end of the extended working channel 22 within the pulmonary airway 5 at the target site 68.

In step 1840, the tool 40 is advanced within the extended working channel 22 to the target site 68. In some embodiments, the tool 40 includes an elongated shaft 84 and a tissue-sampling device 640 disposed at the distal end of the shaft 84. In alternative embodiments, the tool 40 may include a variety of devices at the distal end of the shaft 84. In some embodiments, the shaft 84 may include one or more lumens extending through the length of the shaft 84, or portion of the length of the shaft 84.

During a medical procedure, the extended working channel 22 is inserted into the lungs, directing the locatable guide into the beginning of the last branch of the lungs being navigated to. The end of the extended working channel is flush with the locatable guide, allowing the device to reach the tumor. Once the extended working channel 22 is positioned within the lungs, the balloon portion 24 is inflated with an inflation lumen to create a seal with the vessel wall at the beginning of the last branch being navigated to. In the event that bleeding occurs, the seal between the balloon portion 24 and walls of the lungs prevents any backflow of blood to other areas of the lungs. Instead, if the lungs were to start bleeding at any point during the surgery, the blood will be directed up through the inside of the extended working channel 22 rather than accumulating throughout the lungs. The backflow of blood through the extended working channel 22 would also alert the surgeons of bleeding within the lungs. Alternatively, as described herein above, the extended working channel 22 may be navigated to the source of the bleeding and inflated in order to apply direct pressure to the wound and expedite the coagulation process, thereby stopping the bleeding.

By using the presently-disclosed extended working channel instead of the bronchoscope 14, the surgeon can more easily maneuver into smaller areas of the lung with the assurance that if bleeding does occur, it will be contained, avoiding the risks associated with bleeding of the lungs.

Though the above-described bleeding containment devices and extended working channels including a bleeding containment device are generally described herein with reference to their use within the lungs, they are also suitable for use with a navigation system configured for use in other anatomical luminal networks of a patient.

Although embodiments have been described in detail with reference to the accompanying drawings for the purpose of illustration and description, it is to be understood that the inventive processes and apparatus are not to be construed as limited thereby. It will be apparent to those of ordinary skill in the art that various modifications to the foregoing embodiments may be made without departing from the scope of the disclosure.

What is claimed is:

1. A device for use with a bronchoscope, the device comprising:
an extended working channel configured to provide passage for a tool, wherein the extended working channel and tool are sized to traverse a working channel of the bronchoscope; and
a bleeding containment device associated with a distal end portion of the extended working channel, the bleeding containment device including a balloon portion and an inflation duct associated with the extended working channel.

2. The device of claim 1, wherein the inflation duct is configured to allow fluid to flow to and from the balloon portion for inflation and deflation of the balloon portion.

3. The device of claim 1, wherein extended working channel includes a longitudinally-extending recess defined in an outer surface thereof configured to receive at least a portion of the inflation duct therein.

4. The device of claim 1, wherein the extended working channel includes a recess defined in the distal end portion configured to receive the balloon portion, wherein an outer circumference of the balloon portion is substantially the same as an outer circumference of the extended working channel.

5. A system configured for use in an anatomical luminal network of a patient, comprising:
an extended working channel configured to provide passage for a tool, wherein the extended working channel includes a bleeding containment device including a balloon portion; and
a navigation system for guiding at least one of the tool, the extended working channel, or a locatable guide through a luminal network following a predetermined pathway to target tissue.

6. The system of claim 5, wherein the bleeding containment device further includes an inflation duct associated with the extended working channel and configured to allow fluid to flow to and from the balloon portion for inflation and deflation of the balloon portion.

7. The system of claim 5, wherein the extended working channel includes at least one lumen having open proximal and distal ends, the at least one lumen defining an interior passageway configured to allow slideable movement of the tool between the open proximal and distal ends.

8. The system of claim 7, wherein the at least one lumen includes a first lumen and a second lumen, the first lumen having open proximal and distal ends and defining an interior passageway configured to allow slideable movement of the tool between the open proximal and distal ends.

9. The system of claim 8, wherein the second lumen is configured to allow fluid to flow to and from the balloon portion for inflation and deflation of the balloon portion.

10. The system of claim 9, further comprising a fluid reservoir containing a fluid, wherein the second lumen is fluidly coupled to the fluid reservoir.

11. The system of claim 10, wherein the fluid reservoir includes a temperature control device adapted to maintain the fluid at a predetermined temperature.

12. A biopsy tool for use with an extended working channel, the biopsy tool comprising:
an elongated shaft including a distal end;
a tissue-sampling device disposed at the distal end of the elongated shaft; and
a bleeding containment device associated with a distal end portion of the elongated shaft, the bleeding containment device including a balloon portion and an inflation duct associated with the elongated shaft.

13. The biopsy tool of claim 12, wherein the inflation duct is configured to allow fluid to flow to and from the balloon portion for inflation and deflation of the balloon portion.

14. A method of containing bleeding within the lungs during a medical procedure, comprising:
providing an extended working channel configured to provide passage for a tool, wherein the extended working channel includes a bleeding containment device including a balloon portion;
navigating the extended working channel including the bleeding containment device to a target site within a pulmonary airway of a patient;
inflating the balloon portion, which anchors a distal end of the extended working channel within the pulmonary airway at the target site; and
advancing the tool within the extended working channel to the target site.
